(19) United States
(12) Patent Application Publication
(10) Pub. No.: US 2021/0114953 A1
(43) Pub. Date: Apr. 22, 2021

(54) SYNTHESIS OF E,E-FARNESOL, FARNESYL ACETATE AND SQUALENE FROM FARNESENE VIA FARNESYL CHLORIDE

(71) Applicant: Amyris, Inc., Emeryville, CA (US)

(72) Inventors: Karl Joseph FISHER, Emeryville, CA (US); Frank Xavier WOOLARD, Emeryville, CA (US)

(21) Appl. No.: 16/972,291

(22) PCT Filed: Jun. 7, 2019
(86) PCT No.: PCT/US2019/036064

§ 371 Date: Dec. 4, 2020

Related U.S. Application Data
(60) Provisional application No. 62/682,616, filed on Jun. 8, 2018.

(51) Int. Cl. C07C 1/32; C12P 5/02; C07C 209/60; C07C 17/093; C07C 67/10; C07C 29/128; C07C 315/00; C07C 315/04; A61K 39/39

(52) U.S. Cl. CPC C07C 1/322; C12P 5/026; C07C 209/60; C07C 17/093; A61K 2039/55511; C07C 29/1285; C07C 315/00; C07C 315/04; A61K 39/39; C07C 67/10

(57) ABSTRACT

The present disclosure provides methods for preparing polyunsaturated hydrocarbons, such as E,E-farnesol, farnesyl acetate and squalene, by base catalyzed addition of a dialkylamine to a 3-methylene-1-alkene, such as farnesene. The present disclosure also provides compositions including one more farnesene derivatives prepared using the disclosed methods.

### CROSS-REFERENCES TO RELATED APPLICATIONS

This application claims the benefit of U.S. Provisional Application No. 62/682,616, filed Jun. 8, 2018, which is incorporated by reference in its entirety herein for all purposes.

### BACKGROUND

Farnesene derivatives such as farnesol, farnesyl acetate, and squalene are commercially significant isoprenoid compounds that have found use in a variety of applications. For example, the acyclic sesquiterpene farnesol is used in perfumery as a co-solvent that can regulate the volatility of odorants and emphasize the scent of sweet floral perfumes. Similarly, the acetylation product of farnesol, farnesyl acetate, has also been utilized as a fragrance ingredient. In addition, the alcohol and acetate functional groups of these compounds have allowed them to serve as useful chemical intermediates and building blocks in the synthesis of chemicals based on their isoprenoid polyunsaturated hydrocarbon backbone.

Squalene, another farnesene derivative, is a natural 30-carbon organic compound produced by all animals and plants and originally obtained for commercial purposes primarily from shark liver oil. Because squalene is commonly generated by human sebaceous glands, squalene is often used in cosmetic and personal care products for topical skin lubrication and protection. Squalene also can be an important ingredient in immunological adjuvants to be administered in conjunction with a vaccine. Adjuvants that include squalene can stimulate an immune response with a patient, increasing the response to the vaccine. In some instances, because of this increased response, the amount of antigen included in a vaccine can be reduced by an order of magnitude while still maintaining sufficient immunoprotection. This in turn can result in a 10-fold increase in the number of vaccine doses that can be produced from a given amount of antigen.

While the above farnesene derivative compounds are made naturally in various organisms ranging from microbes to animals, for most of these compounds extraction yields are low and available quantities are far less than are required for many commercial applications. In addition, while some farnesene derivatives can be produced synthetically from petroleum sources, growing concerns related to climate change and sustainability drive a further need for renewable supplies that can help meet global demands while being produced in a more environmentally friendly way. The current disclosure addresses these and other needs.

### BRIEF SUMMARY

Provided herein is a method for preparing a compound of formula (I) having the structure:

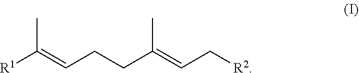

The method includes forming a first reaction mixture including a compound of formula NR³R⁴, a reagent comprising an alkali metal, and a compound of formula (II):

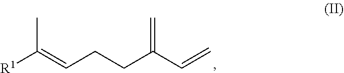

under conditions sufficient to form an amine compound of formula (I) having the structure:

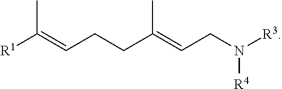

The method further includes forming a second reaction mixture including a chloroformate and the amine compound of formula (I), under conditions sufficient to form a chloride compound of formula (I) having the structure:

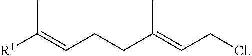

R¹ can be C₂₋₁₈ alkyl or C₂₋₁₈ alkenyl. R² can be NR³R⁴, halogen, OH, —OC(O)R⁵, or —SO₂—R⁵. R³ and R⁴ can each independently be C₁₋₆ alkyl. R⁵ can be C₁₋₆ alkyl, C₃₋₁₀ cycloalkyl, C₃₋₈ heterocycloalkyl, C₆₋₁₂ aryl, or C₅₋₁₂ heteroaryl.

In some embodiments, R³ and R⁴ are each ethyl. In certain aspects, the alkali metal is sodium or lithium. In certain embodiments, the reagent includes an akyllithium compound or an aryllithium compound. In some aspects, the reagent includes n-butyllithium. In some embodiments, the first reaction mixture further includes isopropyl alcohol or styrene. In certain aspects, the chloroformate is isobutyl chloroformate.

In some embodiments, the method further includes forming a third reaction mixture comprising the chloride compound of formula (I) and a compound of formula (III):

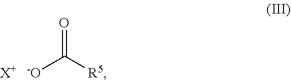

under conditions sufficient to form an ester compound of formula (I) having the structure:

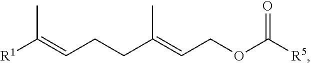

wherein X can be an alkali metal. In certain aspects, the third reaction further includes a crown ether.

In some embodiments, the method further includes forming a fourth reaction mixture comprising a strong base and the ester compound of formula (I) under conditions sufficient to form an alcohol compound of formula (I) having the structure:

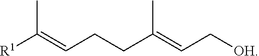

In certain aspects, the strong base includes sodium hydroxide or potassium hydroxide.

In some embodiments, the method further includes forming an alternate third reaction mixture comprising a benzenesulfinate, a quaternary ammonium salt, and the chloride compound of formula (I), under conditions sufficient to form a sulfone compound of formula (I) having the structure:

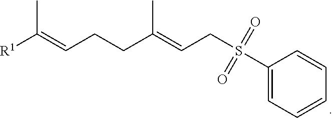

In certain aspects, the benzenesulfinate is sodium benzenesulfinate. In certain embodiments, the quaternary ammonium salt is tetrabutylammonium chloride.

In some embodiments, the method further includes forming an alternate fourth reaction mixture comprising a strong base, the chloride compound of formula (I), and the sulfone compound of formula (I), under conditions sufficient to form a compound of formula (IV) having the structure:

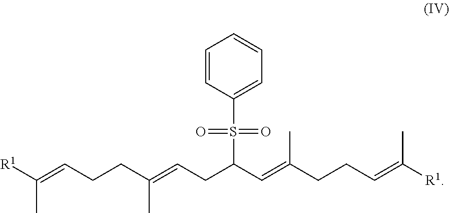

The method can further include forming a fifth reaction mixture including a reducing agent, a palladium catalyst, and a compound of formula (IV), under conditions sufficient to form a compound of formula (I) having the structure:

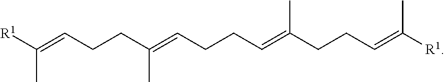

In certain aspects, the fourth reaction mixture further comprises a copper catalyst. In certain embodiments, the copper catalyst is copper iodide. In some aspects, the strong base includes potassium tert-butoxide or sodium hydride. In some embodiments, the reducing agent includes an borohydride reducing agent. In certain aspects, the reducing agent includes lithium In certain embodiments, the reducing agent is lithium triethylborohydride. In some aspects, the palladium catalyst includes palladium chloride. In some embodiments, the palladium catalyst includes [1,2-bis(diphenylphosphino)propane]dichloropalladium(II).

In some embodiments, the compound of formula (II) has the structure:

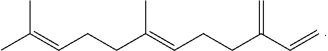

In certain aspects, the method further includes preparing the compound of formula (II) by a process including culturing a microorganism using a carbon source. In certain embodiments, the carbon source is derived from a saccharide. In some embodiments, the amine compound of formula (I) has the structure:

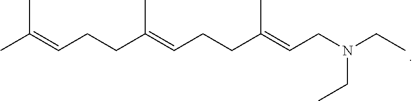 [SMILES: CCN(CC)C/C=C(\C)CC/C=C(\C)CCC=C(C)C]

In some embodiments, the chloride compound of formula (I) has the structure:

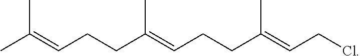 [SMILES: CC(C)=CCC/C(C)=C/CC/C(C)=C/CCl]

In some embodiments, the alcohol compound of formula (I) has the structure:

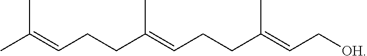 [SMILES: CC(C)=CCC/C(C)=C/CC/C(C)=C/CO]

In some embodiments, the sulfone compound of formula (I) has the structure:

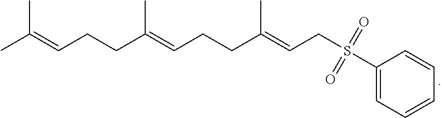 [SMILES: CC(C)=CCC/C(C)=C/CC/C(C)=C/CS(=O)(=O)c1ccccc1]

In some embodiments, the compound of formula (I) has the structure:

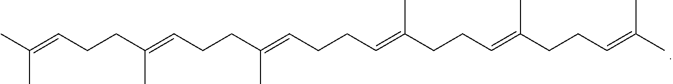 [SMILES: CC(C)=CCC/C(C)=C/CC/C(C)=C/CC/C=C(\C)CC/C=C(\C)CCC=C(C)C]

Also provided is a composition including one or more farnesene derivatives prepared using any of the provided methods as described above. In some embodiments, the composition includes from 0.1 wt % to 3 wt % (2Z,5E)-farnesol relative to the total amount of the one or more farnesene derivatives in the composition. In certain aspects, the composition includes from 0.1 wt % to 99.9 wt % (E,E)-farnesol relative to the total amount the one or more farnesene derivatives in the composition. In certain embodiments, the composition includes from 0.1 wt % to 99.9 wt % farnesyl acetate relative to the total amount of the one or more farnesene derivatives in the composition. In some aspects, the composition includes from 0.1 wt % to 99.9 wt % squalene relative to the total amount of the one or more farnesene derivatives in the composition. In some embodiments, the composition further includes an antigen.

### DETAILED DESCRIPTION

### I. General

The present disclosure provides methods for preparing polyunsaturated hydrocarbons, such as E,E-farnesol, farnesyl acetate and squalene, by base catalyzed addition of a dialkylamine to a 3-methylene-1-alkene, such as farnesene. The present disclosure also provides compositions including one more farnesene derivatives prepared using the disclosed methods.

### II. Definitions

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited, to heptyl, octyl, nonyl, decyl, etc. Alkyl groups can be substituted or unsubstituted.

As used herein, the term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

As used herein, the term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

As used herein, the term “halogen” refers to fluorine, chlorine, bromine and iodine.

As used herein, the term “amine” refers to an —N(R)₂ group where the R groups can be hydrogen, alkyl, alkenyl, alkynyl, cycloalkyl, heterocycloalkyl, aryl, or heteroaryl, among others. The R groups can be the same or different. The amino groups can be primary (each R is hydrogen), secondary (one R is hydrogen) or tertiary (each R is other than hydrogen).

As used herein, the term “cycloalkyl” refers to a saturated or partially unsaturated, monocyclic, fused bicyclic, or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbomene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

As used herein, the term “heterocycloalkyl” refers to a saturated ring system having from 3 to 12 ring members and from 1 to 4 heteroatoms of N, O, and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2-, or 3-pyrrolidine, piperidine can be 1-, 2-, 3-, or 4-piperidine, pyrazolidine can be 1-, 2-, 3-, or 4-pyrazolidine, imidazolidine can be 1-, 2-, 3-, or 4-imidazolidine, piperazine can be 1-, 2-, 3-, or 4-piperazine, tetrahydrofuran can be 1- or 2-tetrahydrofuran, oxazolidine can be 2-, 3-, 4-, or 5-oxazolidine, isoxazolidine can be 2-, 3-, 4-, or 5-isoxazolidine, thiazolidine can be 2-, 3-, 4-, or 5-thiazolidine, isothiazolidine can be 2-, 3-, 4-, or 5-isothiazolidine, and morpholine can be 2-, 3-, or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein, the term “aryl” refers to an aromatic ring system having any suitable number of ring atoms and any suitable number of rings. Aryl groups can include any suitable number of ring atoms, such as 6, 7, 8, 9, 10, 11, 12, 13, 14, 15, or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl, and biphenyl. Some aryl groups have from 6 to 12 ring members, such as phenyl, naphthyl, or biphenyl. Other aryl groups have from 6 to 10 ring members, such as phenyl or naphthyl. Some other aryl groups have 6 ring members, such as phenyl. Aryl groups can be substituted or unsubstituted.

As used herein, the term “heteroaryl” refers to a monocyclic or fused bicyclic or tricyclic aromatic ring assembly containing 5 to 16 ring atoms, where from 1 to 5 of the ring atoms are a heteroatom such as N, O, or S. Additional heteroatoms can also be useful, including, but not limited to, B, Al, Si, and P. The heteroatoms can also be oxidized, such as, but not limited to, —S(O)— and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4-, and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2-, and 3-pyrrole, pyridine includes 2-, 3-, and 4-pyridine, imidazole includes 1-, 2-, 4-, and 5-imidazole, pyrazole includes 1-, 3-, 4-, and 5-pyrazole, triazole includes 1-, 4-, and 5-triazole, tetrazole includes 1- and 5-tetrazole, pyrimidine includes 2-, 4-, 5-, and 6-pyrimidine, pyridazine includes 3- and 4-pyridazine, 1,2,3-triazine includes 4- and 5-triazine, 1,2,4-triazine includes 3-, 5-, and 6-triazine, 1,3,5-triazine includes 2-triazine, thiophene includes 2- and 3-thiophene, furan includes 2- and 3-furan, thiazole includes 2-, 4-, and 5-thiazole, isothiazole includes 3-, 4-, and 5-isothiazole, oxazole includes 2-, 4-, and 5-oxazole, isoxazole includes 3-, 4-, and 5-isoxazole, indole includes 1-, 2-, and 3-indole, isoindole includes 1- and 2-isoindole, quinoline includes 2-, 3-, and 4-quinoline, isoquinoline includes 1-, 3-, and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O, or S, such as pyrrole, pyridine, imidazole, pyrazole, triazole, pyrazine, pyrimidine, pyridazine, triazine (1,2,3-, 1,2,4-, and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4-, and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran, and bipyridine. Still other heteroaryl groups include those having from 5 to 6 ring members and from 1 to 2 ring atoms including N, O, or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4-, and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4-, and 1,3,5-isomers), thiazole, isothiazole, oxazole, isoxazole, quinoxaline, quinazoline, phthalazine, and cinnoline.

As used herein, the term “metal” refers to elements of the periodic table that are metallic and that can be neutral, or negatively or positively charged as a result of having more or fewer electrons in the valence shell than is present for the neutral metallic element. Alkali metals include Li, Na, K, Rb and Cs.

As used herein, the term “borohydride reagent” refers to an organometallic compound with a direct bond between a hydrogen atom and a boron atom. Non-limiting examples of borohydride reagents include sodium borohydride, sodium trialkylborohydride(s), sodium alkoxyborohydride(s), lithium borohydride, lithium trialkylborohydride(s), and lithium alkoxyborohydride(s).

As used herein, the terms “organolithium reagent” and “organolithium compound” refer to an organometallic compound with a direct bond between a carbon atom and a lithium atom. Non-limiting examples of organolithium reagents include vinyllithium, aryllithium (e.g., phenyllithium), and alkyllithium (e.g., n-butyl lithium, sec-butyl lithium, tert-butyl lithium, methyllithium, isopropyllithium or other alkyllithium reagents having 1 to 20 carbon atoms).

As used herein, the term “quaternary ammonium salt” refers to a salt of a positively charged polyatomic ion having the structure NR₄⁺, wherein R is alkyl or aryl.

As used herein, the term “farnesene” refers to α-farnesene, β-farnesene, or a mixture thereof.

As used herein, the term “α-Farnesene” refers to a compound having the following structure:

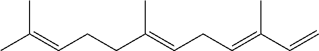

or an isomer thereof. In certain embodiments, the α-farnesene comprises a substantially pure isomer of α-farnesene. In certain embodiments, the α-farnesene comprises a mixture of isomers, such as cis-trans isomers. In further embodiments, the amount of each of the isomers in the α-farnesene mixture is independently from about 0.1 wt % to about 99.9 wt %, from about 0.5 wt % to about 99.5 wt %, from about 1 wt % to about 99 wt %, from about 5 wt % to about 95 wt %, from about 10 wt % to about 90 wt %, or from about 20 wt % to about 80 wt %, based on the total weight of the α-farnesene mixture.

As used herein, the term “β-Farnesene” refers to a compound having the following structure:

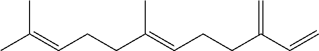

or an isomer thereof. In certain embodiments, the β-farnesene comprises a substantially pure isomer of β-farnesene. In certain embodiments, the β-farnesene comprises a mixture of isomers, such as cis-trans isomers. In further embodiments, the amount of each of the isomers in the β-farnesene mixture is independently from about 0.1 wt % to about 99.9 wt %, from about 0.5 wt % to about 99.5 wt %, from about 1 wt % to about 99 wt %, from about 5 wt % to about 95 wt %, from about 10 wt % to about 90 wt %, or from about 20 wt % to about 80 wt %, based on the total weight of the β-farnesene mixture.

As used herein, the term “farnesol” refers to a compound having the structure:

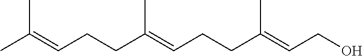 [SMILES: CC(C)=CCC/C(C)=C/CC/C(C)=C/CO]

or an isomer thereof.

As used herein, the term “saccharide” refers to a sugar, such as a monosaccharide, a disaccharide, an oligosaccharide, or a polysaccharide. Monosaccharides include, but are not limited to, glucose, ribose, and fructose. Disaccharides include, but are not limited to, sucrose and lactose. Polysaccharides include, but are not limited to, cellulose, hemicellulose, lignocellulose, and starch. Other saccharides are useful in the present invention.

As used herein, the term “forming a reaction mixture” refers to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture.

As used herein, the term “composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient of a composition must be compatible with the other ingredients of a formulation composition and not deleterious to the recipient thereof.

### III. Methods

Disclosed herein are synthetic routes to polyunsaturated hydrocarbons such as industrially relevant farnesene derivatives. The synthetic routes provide advantageous alternate supplies of chemical products and intermediates that are conventionally isolated as natural products, or created from non-renewable petroleum-based feedstocks. The provided methods can employ renewable starting materials such as carbon sources fed to microbial cultures, and can be readily applied to industrial scale processes.

The present disclosure provides several methods of preparing compounds having the structure of formula (I):

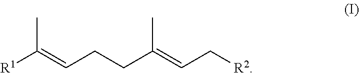

R¹ of formula (I) can be hydrogen, C₂₋₁₈ alkyl, or C₂₋₁₈ alkenyl. R² of formula (I) can be NR³R⁴, halogen, OH, —OC(O)R⁵, or —SO₂—R⁵. R³ and R⁴ can each independently be C₁₋₆ alkyl. R⁵ can be C₁₋₆ alkyl, C₃₋₁₀ cycloalkyl, C₃₋₈ heterocycloalkyl, C₆₋₁₂ aryl, or C₅₋₁₂ heteroaryl. The methods include forming a first reaction mixture including a compound of formula NR³R⁴, a strong base, and a compound of formula (II):

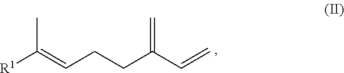

under conditions sufficient to form an amine compound of formula (I) having the structure:

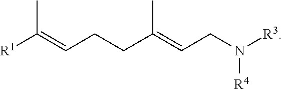

The methods further include forming a second reaction mixture including a chloroformate and the amine compound of formula (I), under conditions sufficient to form a chloride compound of formula (I) having the structure:

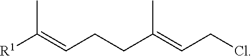

R¹ of formula (I) can be C₂₋₁₈ alkyl or C₂₋₁₈ alkenyl. In some embodiments, R¹ is C₂₋₁₀ alkenyl, e.g., C₂₋₆ alkenyl, C₃₋₇ alkenyl, C₄₋₈ alkenyl, C₅₋₉ alkenyl, or C₆₋₁₀ alkenyl. R¹ can be, for example, ethenyl, propenyl, butenyl, pentenyl, or hexenyl. In some embodiments, R¹ is a branched hydrocarbon. In some embodiments, R¹— is 2-methylpent-2-ene.

R³ and R⁴ of formula (I) can each independently be C₁₋₆ alkyl, e.g., C₁₋₃ alkyl, C₂₋₄ alkyl, C₃₋₅ alkyl, or C₄₋₆ alkyl. In some embodiments, R³ is methyl, ethyl, or propyl. In some embodiments, R⁴ is methyl, ethyl, or propyl. In some embodiments, R³ and R⁴ are each ethyl.

The strong base of the first reaction mixture can be a reagent including an alkali metal. In some embodiments, the alkali metal is sodium, lithium, or potassium. In certain aspects, the strong base is sodium metal or lithium metal. In some embodiments, the strong base includes potassium hydroxide, potassium tert-butoxide, or sodium hydroxide. In some embodiments, the reagent includes an organolithium compound. The organolithium compound can be, for example, an alkyllithium compound or an aryllithium compound. In some embodiments, the strong base of the first reaction mixture includes an alkyllithium compound. In some embodiments, the strong base includes n-butyllithium, sec-butyllithium, or tert-butyllithium.

In some embodiments, the chloroformate of the second reaction mixture is an alkyl chloroformate. For example, the chloroformate can be methyl chloroformate, ethyl chloroformate, propyl chloroformate, isopropyl chloroformate, butyl chloroformate, sec-butyl chloroformate, isobutyl chloroformate, or tert-butyl chloroformate. In some embodiments, the chloroformate is an aryl chloroformate. For example, the chloroformate can be phenyl chloroformate. In some embodiments, the chloroformate is isobutyl chloroformate.

In certain aspects, the first reaction mixture further includes an organic solvent. In some embodiments, the organic solvent includes isopropyl alcohol. In some embodiments, the organic solvent includes styrene.

The provided methods can further include forming a third reaction mixture including the chloride compound of formula (I) and a compound of formula (III):

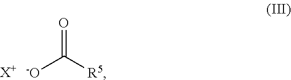

under conditions sufficient to form an ester compound of formula (I) having the structure:

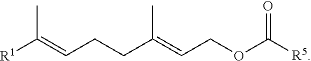

X of formula (III) can be an alkali metal. R⁵ can be C₁₋₆ alkyl, C₃₋₁₀ cycloalkyl, C₃₋₈ heterocycloalkyl, C₆₋₁₂ aryl, or C₅₋₁₂ heteroaryl.

X of formula (III) can be an alkali metal. In some embodiments, X is lithium, sodium, or potassium. R⁵ can be C₁₋₆ alkyl, C₃₋₁₀ cycloalkyl, C₃₋₈ heterocycloalkyl, C₆₋₁₂ aryl, or C₅₋₁₂ heteroaryl. In some embodiments, R⁵ is C₁₋₆ alkyl, e.g., C₁₋₃ alkyl, C₂₋₄ alkyl, C₃₋₅ alkyl, or C₄₋₆ alkyl. In certain aspects, R⁵ is methyl, ethyl, or propyl. In some embodiments, R⁵ is methyl. In some embodiments, the compound of formula (III) is potassium acetate.

In certain aspects, the third reaction can further include a crown ether. The crown ether can be a cyclic oligomer of ethylene oxide. In some embodiments, the crown ether is 12-crown-4, 15-crown-5, 18-crown-6, dibenzo-18-crown-6, or diaza-18-crown-6. In certain aspects, the crown ether is 18-crown-6.

The provided methods can further include forming a fourth reaction mixture comprising a strong base and the ester compound of formula (I) under conditions sufficient to form an alcohol compound of formula (I) having the structure:

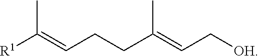

The strong base of the fourth reaction mixture can be a reagent including an alkali metal. In some embodiments, the alkali metal is sodium, lithium, or potassium. In certain aspects, the strong base is sodium metal or lithium metal. In some embodiments, the strong base includes potassium hydroxide, potassium tert-butoxide, or sodium hydroxide. In some embodiments, the reagent includes an organolithium compound. The organolithium compound can be, for example, an alkyllithium compound or an aryllithium compound. In some embodiments, the strong base of the fourth reaction mixture includes an alkyllithium compound. In some embodiments, the strong base includes n-butyllithium, sec-butyllithium, or tert-butyllithium.

In some embodiments, the methods include forming an alternative third reaction mixture that includes a benzenesulfinate, a quaternary ammonium salt, and the chloride compound of formula (I) under conditions sufficient to form a sulfone compound of formula (I) having the structure:

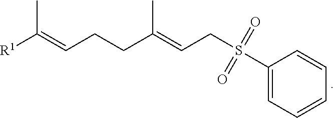

The benzenesulfinate of the third reaction mixture can be a salt. In some embodiments, the benzenesulfinate is sodium benzenesulfinate. The quaternary ammonium salt of the third reaction mixture can include alkyl or aryl groups connected to its nitrogen atom. Each of the groups of the quaternary ammonium salt can be the same as, or different from, one or more other groups of the salt. In some embodiments, the quaternary ammonium salt includes a halogen. In certain aspects, the quaternary ammonium salt includes bromine. In some embodiments, the quaternary ammonium salt is tetrabutylammonium bromide.

In some embodiments in which the methods include forming an alternative third reaction mixture as described above, the methods further include forming an alternative fourth reaction mixture including a strong base, the chloride compound of formula (I), and the sulfone compound of formula (I), under conditions sufficient to form a compound of formula (IV) having the structure:

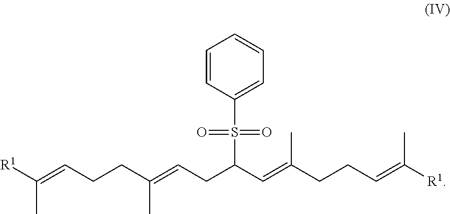

In some embodiments, the alternative fourth reaction mixture further includes a copper catalyst. In certain aspects, the copper catalyst includes a halogen. In some embodiments, the copper catalyst includes copper iodide.

The strong base of the alternative fourth reaction mixture can be a reagent including an alkali metal. In some embodiments, the alkali metal is sodium, lithium, or potassium. In certain aspects, the strong base is sodium metal or lithium metal. In some embodiments, the strong base includes potassium hydroxide, potassium tert-butoxide, or sodium hydride. In some embodiments, the reagent includes an organolithium compound. The organolithium compound can be, for example, an alkyllithium compound or an aryllithium compound. In some embodiments, the strong base of the alternative fourth reaction mixture includes an alkyllithium compound. In some embodiments, the strong base includes n-butyllithium, sec-butyllithium, or tert-butyllithium.

In some embodiments, in which the methods include forming an alternative fourth reaction mixture as described above, the methods further include forming a fifth reaction mixture including a reducing agent, a palladium catalyst, and a compound of formula (IV), under conditions sufficient to form a compound of formula (I) having the structure:

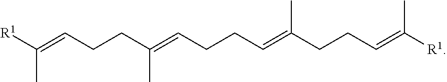

In some embodiments, the palladium catalyst of the fifth reaction mixture includes a halogen. In certain aspects, the palladium catalyst includes palladium chloride. In some embodiments, the palladium catalyst includes [1,2-bis(diphenylphosphino)propane]dichloropalladium(II).

The reducing agent of the fifth reaction mixture can include a borohydride reducing agent. The borohydride reducing agent can include one or more alkyl, alkoxy, or aryl groups. Each of the alkyl, alkoxy, or aryl groups of the borohydride reducing agent can be the same as, or different from, one or more other groups of the borohydride reducing agent. In some embodiments, the borohydride reducing agent includes three alkyl groups. In some embodiments, the borohydride reducing agents includes triethylborohydride. In certain aspects, the reducing agent includes an alkali metal. In some embodiments, the reducing agent includes lithium. In some embodiments, the reducing agent includes lithium metal in ethylamine. In some embodiments, the reducing agent includes lithium triethylborohydride.

In some embodiments, the compound of formula (II) is farnesene having the structure:

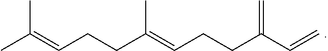

In some embodiments, the amine compound of formula (I) is (N,N)-diethylfarnesylamine having the structure:

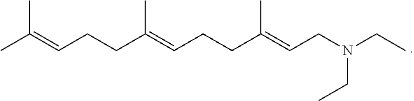 [SMILES: CCN(CC)C/C=C(\C)CC/C=C(\C)CCC=C(C)C]

In some embodiments, the chloride compound of formula (I) is (E,E)-farnesyl chloride having the structure:

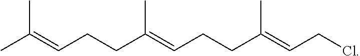 [SMILES: CC(C)=CCC/C(C)=C/CC/C(C)=C/CCl]

In some embodiments, the ester compound of formula (I) is (E,E)-farnesyl acetate having the structure:

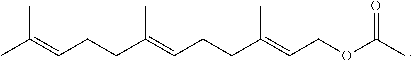 [SMILES: CC(=O)OC/C=C(\C)CC/C=C(\C)CCC=C(C)C]

In some embodiments, the alcohol compound of formula (I) is (E,E)-farnesol having the structure:

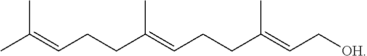 [SMILES: CC(C)=CCC/C(C)=C/CC/C(C)=C/CO]

In some embodiments, the sulfone compound of formula (I) is (E,E)-farnesyl phenyl sulfone having the structure:

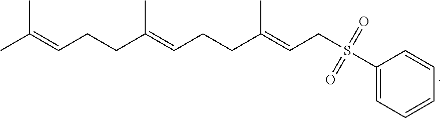 [SMILES: CC(C)=CCC/C(C)=C/CC/C(C)=C/CS(=O)(=O)c1ccccc1]

In some embodiments, the compound of formula (I) is squalene having the structure:

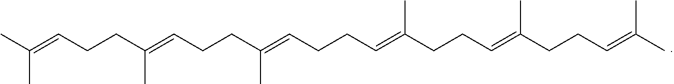 [SMILES: CC(C)=CCC/C(C)=C/CC/C(C)=C/CC/C=C(\C)CC/C=C(\C)CCC=C(C)C]

In certain aspects, the compound of formula (II) is farnesene. Farnesene is a sesquiterpene which are part of a larger class of compound called terpenes. A large and varied class of hydrocarbons, terpenes include hemiterpenes, monoterpenes, sesquiterpenes, diterpenes, sesterterpenes, triterpenes, tetraterpenes, and polyterpenes. As a result, the farnesene can be isolated or derived from terpene oils to produce the derivatives of the provided methods and compositions. In some embodiments, the farnesene is derived from a chemical source (e.g., petroleum or coal) or obtained by a chemical synthetic method. In other embodiments, the farnesene is prepared by fractional distillation of petroleum or coal tar. In further embodiments, the farnesene is prepared by any known chemical synthetic method.

In certain embodiments, the farnesene is derived from a biological source. In other embodiments, the farnesene can be obtained from a readily available, renewable carbon source. In further embodiments, the farnesene is prepared by contacting a cell capable of making a farnesene with a carbon source under conditions suitable for making the farnesene.

In some embodiments, the provided methods include preparing the compound of formula (II), e.g., farnesene, by a process that includes culturing a microorganism using a carbon source. For example, farnesene can be prepared by culturing wild-type, evolved, or genetically modified microbial host cells selected or designed for their ability to synthesize the isoprenoid compound. Any suitable microbial host cell can be genetically modified to make farnesene. A genetically modified host cell is one in which nucleic acid molecules have been inserted, deleted or modified (i.e., mutated; e.g., by insertion, deletion, substitution, and/or inversion of nucleotides), to produce farnesene. Illustrative examples of suitable host cells include any archae, bacterial, or eukaryotic cell. Examples of archae cells include, but are not limited to those belonging to the genera: Aeropyrum, Archaeglobus, Halobacterium, Methanococcus, Methanobacterium, Pyrococcus, Sulfolobus, and Thermoplasma. Illustrative examples of archae species include but are not limited to: Aeropyrum pernix, Archaeoglobus fulgidus, Methanococcus jannaschii, Methanobacterium thermoautotrophicum, Pyrococcus abyssi, Pyrococcus horikoshii, Thermoplasma acidophilum, and Thermoplasma volcanium. Examples of bacterial cells include, but are not limited to those belonging to the genera: Agro bacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas.

Illustrative examples of bacterial species include but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella jlexneri, Shigella sonnei, Staphylococcus aureus, and the like.

In general, if a bacterial host cell is used, a non-pathogenic strain is preferred. Illustrative examples of species with nonpathogenic strains include but are not limited to: Bacillus subtilis, Escherichia coli, Lactibacillus acidophilus, Lactobacillus helveticus, Pseudomonas aeruginosa, Pseudomonas mevalonii, Pseudomonas pudita, Rhodobacter sphaeroides, Rodobacter capsulatus, Rhodospirillum rubrum, and the like.

Examples of eukaryotic cells include but are not limited to fungal cells. Examples of fungal cells include, but are not limited to those belonging to the genera: Aspergillus, Candida, Chrysosporium, Cryotococcus, Fusarium, Kluyveromyces, Neotyphodium, Neurospora, Penicillium, Pichia, Saccharomyces, Trichoderma and Xanthophyllomyces (formerly Phajfia).

Illustrative examples of eukaryotic species include but are not limited to: Aspergillus nidulans, Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phajfia rhodozyma).

In general, if a eukaryotic cell is used, a non-pathogenic strain is preferred. Illustrative examples of species with nonpathogenic strains include but are not limited to: Fusarium graminearum, Fusarium venenatum, Pichia pastoris, Saccaromyces boulardi, and Saccaromyces cerevisiae.

In some embodiments, the host cells of the present invention have been designated by the Food and Drug Administration as GRAS or Generally Regarded As Safe. Illustrative examples of such strains include: Bacillus subtilis, Lactibacillus acidophilus, Lactobacillus helveticus, and Saccharomyces cerevisiae.

Any carbon source that can be converted into farnesene can be used herein. In some embodiments, the carbon source is a sugar or a non-fermentable carbon source. The sugar can be any sugar known to those of skill in the art. In certain embodiments, the sugar is a monosaccharide, disaccharide, polysaccharide or a combination thereof. In other embodiments, the sugar is a simple sugar (e.g., a monosaccharide or a disaccharide). Some non-limiting examples of suitable monosaccharides include glucose, galactose, mannose, fructose, ribose, and combinations thereof. Some non-limiting examples of suitable disaccharides include sucrose, lactose, maltose, trehalose, cellobiose, and combinations thereof. In still other embodiments, the simple sugar is sucrose. In certain embodiments, the farnesene can be obtained from a polysaccharide. Some non-limiting examples of suitable polysaccharides include starch, glycogen, cellulose, chitin and combinations thereof.

The sugar suitable for making the farnesene can be found in a wide variety of crops or sources. Some non-limiting examples of suitable crops or sources include sugar cane, bagasse, miscanthus, sugar beet, sorghum, grain sorghum, switchgrass, barley, hemp, kenaf, potatoes, sweet potatoes, cassava, sunflower, fruit, molasses, whey or skim milk, corn, stover, grain, wheat, wood, paper, straw, cotton, many types of cellulose waste, and other biomass. In certain embodiments, the suitable crops or sources include sugar cane, sugar beet and corn. In other embodiments, the sugar source is cane juice or molasses.

A non-fermentable carbon source is a carbon source that cannot be converted by the organism into ethanol. Some non-limiting examples of suitable non-fermentable carbon sources include acetate and glycerol. In certain embodiments, the farnesene can be prepared in a facility capable of biological manufacture of farnesene. The facility can include any structure useful for preparing farnesene using a microorganism. In some embodiments, the biological facility includes one or more of the cells disclosed herein. In further embodiments, the biological facility includes a fermentor holding one or more cells described herein. Any fermentor that can provide cells or bacteria a stable environment in which they can grow or reproduce can be used herein.

### IV. Compositions

Also disclosed herein are compositions that include one or more polyunsaturated hydrocarbons produced using the provided methods described above. In some embodiments, the compositions include one or more farnesene derivatives prepared using any of the provided methods. In some embodiments, the compositions include (E,E)-farnesol produced using the provided methods described above. The concentration of (E,E)-farnesol relative the total amount of the one or more farnesene derivatives in the composition can, for example, be from 0.1 wt % to 99.9 wt %, e.g., from 0.1 wt % to 60 wt %, from 10 wt % to 70 wt %, from 20 wt % to 80 wt %, from 30 wt % to 90 wt %, or from 40 wt % to 99.9 wt %. In terms of upper limits, the (E,E)-farnesol concentration relative to that of the other farnesene derivatives can be less than 99.9 wt %, e.g., less than 90 wt %, less than 80 wt %, less than 70 wt %, less than 60 wt %, less than 50 wt %, less than 40 wt %, less than 30 wt %, less than 20 wt %, or less than 10 wt %. In terms of lower limits, the (E,E)-farnesol concentration relative to that of the other farnesene derivatives can be greater than 0.1 wt %, e.g., greater than 10 wt %, greater than 20 wt %, greater than 30 wt %, greater than 40 wt %, greater than 50 wt %, greater than 60 wt %, greater than 70 wt %, greater than 80 wt %, or greater than 90 wt %. Higher concentrations, e.g., greater than 99.9 wt %, and lower concentrations, e.g., less than 0.1 wt %, are also contemplated.

As used herein, the term “total amount of the one or more farnesene derivatives” refers to the combined quantity of derivatives that can include dihydrofarnesene, tetrahydrofarnesene, hexahydrofarnesene, farnesane, and multimers thereof as well as multimers of farnesene. Farnesene derivatives can further include reactive derivatives of farnesene and/or farnesane. These include oxidative derivatives. hydroxyl derivatives such as farnesol, epoxy derivatives, and other derivatives of farnesene and/or farnesane recognized by those skilled in the art. In some embodiments, farnesene derivatives can also include partially hydrogenated farnesene.

In some embodiments, the compositions include farnesyl acetate produced using the provided methods described above. The concentration of farnesyl acetate relative the total amount of the one or more farnesene derivatives in the composition can, for example, be from 0.1 wt % to 99.9 wt %, e.g., from 0.1 wt % to 60 wt %, from 10 wt % to 70 wt %, from 20 wt % to 80 wt %, from 30 wt % to 90 wt %, or from 40 wt % to 99.9 wt %. In terms of upper limits, the farnesyl acetate concentration relative to that of the other farnesene derivatives can be less than 99.9 wt %, e.g., less than 90 wt %, less than 80 wt %, less than 70 wt %, less than 60 wt %, less than 50 wt %, less than 40 wt %, less than 30 wt %, less than 20 wt %, or less than 10 wt %. In terms of lower limits, the farnesyl acetate concentration relative to that of the other farnesene derivatives can be greater than 0.1 wt %, e.g., greater than 10 wt %, greater than 20 wt %, greater than 30 wt %, greater than 40 wt %, greater than 50 wt %, greater than 60 wt %, greater than 70 wt %, greater than 80 wt %, or greater than 90 wt %. Higher concentrations, e.g., greater than 99.9 wt %, and lower concentrations, e.g., less than 0.1 wt %, are also contemplated.

In some embodiments, the compositions include squalene produced using the provided methods described above. The concentration of squalene relative the total amount of the one or more farnesene derivatives in the composition can, for example, be from 0.1 wt % to 99.9 wt %, e.g., from 0.1 wt % to 60 wt %, from 10 wt % to 70 wt %, from 20 wt % to 80 wt %, from 30 wt % to 90 wt %, or from 40 wt % to 99.9 wt %. In terms of upper limits, the squalene concentration relative to that of the other farnesene derivatives can be less than 99.9 wt %, e.g., less than 90 wt %, less than 80 wt %, less than 70 wt %, less than 60 wt %, less than 50 wt %, less than 40 wt %, less than 30 wt %, less than 20 wt %, or less than 10 wt %. In terms of lower limits, the squalene concentration relative to that of the other farnesene derivatives can be greater than 0.1 wt %, e.g., greater than 10 wt %, greater than 20 wt %, greater than 30 wt %, greater than 40 wt %, greater than 50 wt %, greater than 60 wt %, greater than 70 wt %, greater than 80 wt %, or greater than 90 wt %. Higher concentrations, e.g., greater than 99.9 wt %, and lower concentrations, e.g., less than 0.1 wt %, are also contemplated.

A consequence of the disclosed synthetic processes is that farnesene derivatives thus produced can include one or more isomers or other impurities characteristic of its production process. For example, farnesol made with the provided process can include a small amount of double-bond 2Z isomer. This isomer generally is not present in farnesol isolated as a natural product. The concentration of (2Z,5E)-farnesol relative the total amount of the one or more farnesene derivatives in the composition can, for example, be from 0.1 wt % to 3 wt %, e.g., from 0.1 wt % to 1.8 wt %, from 0.4 wt % to 2.1 wt %, from 0.7 wt % to 2.4 wt %, from 1 wt % to 2.7 wt %, or from 1.3 wt % to 3 wt %. In terms of upper limits, the (2Z,5E)-farnesol concentration relative to that of the other farnesene derivatives can be less than 3 wt %, e.g., less than 2.7 wt %, less than 2.4 wt %, less than 2.1 wt %, less than 1.8 wt %, less than 1.5 wt %, less than 1.2 wt %, less than 0.9 wt %, less than 0.6 wt %, or less than 0.3 wt %. In terms of lower limits, the (2Z,5E)-farnesol concentration relative to that of the other farnesene derivatives can be greater than 0.1 wt %, e.g., greater than 0.4 wt %, greater than 0.7 wt %, greater than 1 wt %, greater than 1.3 wt %, greater than 1.6 wt %, greater than 1.9 wt %, greater than 2.2 wt %, greater than 2.5 wt %, or greater than 2.8 wt %. Higher concentrations, e.g., greater than 3 wt %, and lower concentrations, e.g., less than 0.1 wt %, are also contemplated.

In some embodiments, the compositions further include an antigen. The antigen can be any molecule capable of inducing an immune response in a host organism or subject. In certain aspects, the antigen includes a polysaccharide or at least a fragment thereof. In certain aspects, the antigen includes a lipid or at least a fragment thereof. In certain aspects, the antigen includes a protein or at least a fragment thereof. Examples include, but are not limited to, viral proteins, bacterial proteins, parasite proteins, cytokines, chemokines, immunoregulatory agents, and therapeutic agents. The antigen can be a wild-type protein, a truncated form of that protein, a mutated form of that protein, or any other variant of that protein, in each case capable of contributing to immune responses upon expression in the animal or human host. In some embodiments, the antigen is in an immunogenic form as a vaccine.

While the processes and systems provided herein have been described with respect to a limited number of embodiments, the specific features of one embodiment should not be attributed to other embodiments of the processes or systems. No single embodiment is representative of all aspects of the methods or systems. In certain embodiments, the processes can include numerous steps not mentioned herein. In certain embodiments, the processes do not include any steps not enumerated herein. Variations and modifications from the described embodiments exist.

All publications and patent applications mentioned in this specification are herein incorporated by reference to the same extent as if each individual publication or patent application was specifically and individually indicated to be incorporated by reference. Although the claimed subject matter has been described in some detail by way of illustration and example for purposes of clarity of understanding, it will be readily apparent to those of ordinary skill in the art in light of the teachings herein that certain changes and modifications may be made thereto without departing from the spirit or scope of the appended claims.

### V. Examples

The present disclosure will be better understood in view of the following non-limiting examples. In the following examples, efforts have been made to ensure accuracy with respect to numbers used (for example, amounts, temperature, and so on), but variation and deviation can be accommodated, and in the event a clerical error in the numbers reported herein exists, one of ordinary skill in the arts to which this invention pertains can deduce the correct amount in view of the remaining disclosure herein. Unless indicated otherwise, temperature is reported in degrees Celsius, and pressure is at or near atmospheric pressure at sea level. All reagents, unless otherwise indicated, were obtained commercially. The following examples are intended for illustrative purposes only and do not limit in any way the scope of the present invention.

### Example 1. N,N-Diethylfarnesylamine

Diethylamine (285 ml, 2.74 moles) was added to a 3-liter flask, and sodium metal (4.84 g, 0.21 moles) was added in four portions followed by 1.6 mL 2-propanol. The mixture was heated to reflux and farnesene (418.9 g, 2.05 moles) was added dropwise over 1 hour. At the end of the addition, the internal temperature of the mixture had risen to 76° C. After twenty additional minutes, the internal temperature had risen to 103° C. and the heater was turned off. After allowing the mixture to rest overnight, gas chromatography analysis showed that the reaction had achieved approximately 85% conversion. An additional 1.3 g sodium metal and 60 mL diethylamine was added and the mixture was heated for three hours. The cooled reaction mixture was washed with 100 mL 5% potassium carbonate solution. The lower aqueous phase was separated and discarded. The organic material was concentrated by rotary evaporation and distilled on a Kugelrohr apparatus at a boiling point of 210° C. and a pressure of 0.9 torr to yield N,N-diethylfarnesylamine as a yellow liquid (509.3 g, 90%). Proton NMR: 5.26 (t, 1H), 5.08 (q, 2H), 3.06 (d, 2H), 2.51 (q, 4H), 1.8-2.7 (m, 8H), 1.68 (bs, 3H), 1.64 (bs, 3H), 1.60 (bs, 3H), 1.03 (t, 6H).

### Example 2. N,N-Diethylfarnesylamine

Styrene (5.8 ml, 0.051 moles) was added to diethylamine (53 ml, 0.51 moles), followed by five portions of lithium wire (0.35 g total, 0.050 moles). The mixture was heated for 4 hours at 60° C. to dissolve most of the lithium, at which time farnesene (86.9 g, 0.425 moles) was added. After 20 hours at 60° C., gas chromatography analysis showed good conversion, and the mixture was cooled to room temperature. The mixture was then filtered, and volatile impurities were removed by rotary evaporation. The resulting yellow oil was diluted in 150 mL hexanes and washed with 60 mL of a 10% potassium carbonate solution. The organic phase was dried over anhydrous potassium carbonate, filtered and concentrated. The product was distilled on a Kugelrohr apparatus at a boiling point of 150-165° C. and a pressure of 0.3 torr to yield N,N-diethylfarnesylamine (106.9 g, 90.6%).

### Example 3. E,E-Farnesyl Chloride

N,N-diethylfarnesylamine (13.4 g, 48.4 mmol) was diluted in 40 mL toluene. The solution was cooled in an ice water bath and isobutyl chloroformate (6.3 ml, 48.4 mmol) was added dropwise. After stirring for 2 hours at room temperature (25° C.), has chromatography analysis showed high conversion. After allowing the solution to stand at room temperature, a small amount of solid impurity was removed by filtration and the solvent was removed by rotary evaporation. The N,N-diethyl isobutyl carbamate byproduct was removed by distillation at reduced pressure at reduced pressure to result in 11.8 g light brown oil at nearly quantitative yield. Proton NMR: 5.45 (t, 1H), 5.09 (t, 2H), 4.10 (d, 2H), 2.05-2.15 (m, 6H), 1.93-2.03 (m, 2H), 1.73 (bs, 3H), 1.67 (bs, 3H), 1.60 (bs, 6H).

### Example 4. Farnesyl Acetate

Farnesyl chloride (11.8 g, 48.4 mmol) was diluted in 60 mL acetonitrile. Solid potassium acetate (5.76 g, 58.7 mmol) was added, followed by 0.51 g 18-crown-6. The mixture was heated to reflux for 3 hours. Solvent was removed by rotary evaporation and the residue was dissolved in a mixture of 30 mL ethyl acetate and 20 mL water. The organic phase was separated, dried oved solid potassium carbonate, filtered and concentrated to yield 13.15 g oil.

### Example 5. Farnesyl Acetate

Farnesyl chloride (1.66 g, 6.9 mmol) was dissolved in 11 mL acetonitrile and solid potassium acetate (0.96 g, 9.8 mmol) was added followed by 133 mg 18-crown-6. The resulting suspension was heated at 75° C. for 70 minutes. After cooling the suspension, the majority of the acetonitrile was removed by rotary evaporation. Crude acetate was recovered by dilution with 20 mL water and 20 mL hexanes, and separation of the organic phase. The aqueous phase was extracted with an additional 10 mL hexanes and the combined organics were concentrated. The ester was filtered through silica gel using 5% ethyl acetate as eluent to yield the desired ester as a colorless oil (1.69 g, 87%). Proton NMR: 5.35 (dt, 1H), 5.08 (m, 2H), 4.59 (d, 2H), 2.03-2.15 (m, 6H), 1.94-2.02 (m, 2H), 1.71 (bs, 3H), 1.68 (bs, 3H), 1.60 (bs, 6H).

### Example 6. E,E-farnesol

Farnesyl acetate (227.7 mg, 0.8625 mmol) was diluted in 2 mL methanol to which 2 mL of a solution of 5% sodium hydroxide in methanol was added. After 4 hours, solid potassium hydroxide was added and the mixture was heated at reflux for 20 hours. The mixture was treated with 10 mL saturated ammonium chloride and 10 mL water, and then extracted twice with 15 mL ethyl acetate. The combined organic solutions were dried over potassium carbonate, filtered, and concentrated to yield 185.2 mg yellow brown oil. The oil was purified by silica gel chromatography using a step gradient from 15% ethyl acetate/hexanes to 20% ethyl acetate/hexanes. Fractions were combined to give 163.1 mg (85%) desired product at a purity of 98% as measured by gas chromatography-mass spectrometry area percent. Proton NMR: 5.42 (dt, 1H), 5.09 (q, 2H), 4.15 (t, 2H), 1.95-2.15 (m, 8H), 1.68 (bs, 6H), 1.60 (bs, 6H).

### Example 7. E,E-farnesol

Farnesyl chloride (11.66 g, 0.0486 mol) was diluted with 110 mL acetonitrile, to which solid potassium acetate (9.6 g, 0.0978 moles) and 18-crown-6 were added. The reaction was heated at 75° C. for 70 minutes. After cooling the suspension, the acetonitrile was removed by rotary evaporation. A solution of potassium hydroxide in methanol was prepared by dissolving 6.8 g of KOH in 136 mL of methanol. To the crude farnesyl acetate intermediate was added 2.5 equivalents of the potassium hydroxide solution. The suspension was stirred at 25° C. overnight. Methanol was then removed by rotary evaporation, and the residue was diluted with 200 mL water, and then extracted with 200 mL hexanes. The aqueous phase was separated and extracted with an additional 100 mL hexanes. The combined hexane layers were concentrated and the E,E-farnesol was purified by Kugelrohr distillation at a boiling point of 150° C. and a pressure of 0.1 mm Hg to yield the E,E-farnesol (10.31 g, 95.6%).

### Example 8. E,E-Farnesyl Phenyl Sulfone Method 1

Tetrahydrofuran (170 mL), farnesyl chloride (10.0 g, 41.5 mmol), sodium benzene sulfinate (10.2 g, 62.3 mmol) and tetrabutylammonium bromide (1.34 g, 4.15 mmol) were added to a 500-mL three necked round bottom flask equipped with a heating mantle, magnetic stirrer, reflux condenser, glass stopper and nitrogen inlet. The resulting mixture was then refluxed for 5 days. Solid was removed by vacuum filtration, and the solvent was removed at reduced pressure. Additional impurities were removed by distillation using a Kugelrohr apparatus at a boiling point of 150° C. for 2 hours. The product was further purified by silica gel filtration gel with 30% ethyl acetate to remove some brown color, yielding E,E-farnesyl phenyl sulfone as a light yellow orange oil (7.24 g, 50.3%). Proton NMR: 7.87 (dd, 2H), 7.62 (t, 1H), 7.54 (t, 2H), 5.19 (t, 1H), 5.03-5.10 (m, 2H), 3.81 (d, 2H), 1.93-2.10 (m, 8H), 1.68 (bs, 3H), 1.60 (bs, 3H), 1.58 (bs, 3H), 1.31 (bs, 3H).

### Example 9. E,E-Farnesyl Phenyl Sulfone Method 2

Crude farnesyl chloride (30.7 g, 64% pure, 82.5 mmol) was diluted in 150 mL THF to which tetrabutylammonium bromide (1.91 g) and benzene sulfinic acid sodium salt (16.1 g, 105.9 mmol) were added. After heating for 2 hours at 60° C., gas chromatography analysis showed approximately 75% conversion. The mixture was then heated at 60° C. for an additional 3.5 hours before being allowed to cool to room temperature. Most of the solvent was removed by rotary evaporation and the residue was dissolved in 100 mL ethyl acetate and 100 mL water. The organic phase was separated and concentrated by rotary evaporation to give 32.3 g nearly colorless oil. Kugelrohr distillation (0.6 torr, 90° C.) yielded 10.9 g distillate including the carbamate byproduct and some Cis hydrocarbon impurities. The product was further purified by silica gel column chromatography using a 10% ethyl acetate to 20% ethyl acetate/hexanes step gradient, followed by evaporation and drying to give 17.1 g desired farnesyl phenyl sulfone in 81% yield.

### Example 10. Squalene Phenyl Sulfone Method 1

An oven dried 250-mL three necked round bottom flask and pressure equalizing addition funnel were assembled while hot, cooled under argon, and then equipped with a thermometer, rubber septum, and magnetic stirrer. The flask was charged with farnesyl phenyl sulfone (2.94 g, 8.48 mmol), farnesyl chloride (2.45 g, 10.2 mmol), copper (I) iodide (162 mg, 0.848 mmol) and 75 mL of dry tetrahydrofuran. The mixture was stirred and cooled to −45° C. by partial immersion in a dry ice/acetone bath. A solution of potassium tert-butoxide (1.55 g, 12.7 mmol) in 15 mL tetrahydrofuran was added dropwise over 15 minutes and the stirring was continued at a temperature of −45° C. to −50° C. for 2 hours. At the end of this time, thin layer chromatography analysis showed the sulfone to be gone. The mixture was allowed to warm to room temperature. The tetrahydrofuran was removed under reduced pressure and the resulting dark residue was dissolved in 100 mL diethyl ether. This solution was then extracted with 0.3% aqueous HCl (2×40 mL), water (2×40 mL), and brine (40 mL). The organic phase was dried over magnesium sulfate, the solution was filtered, and the solvent was removed under reduced pressure to afford 4.93 g of an orange oil. The product was purified by silica gel chromatography using a 4.5 cm×27 cm column with 20% ethyl acetate in hexanes to yield squalene phenyl sulfone as a yellow orange oil (3.86 g, 83.42%). Proton NMR: 7.85 (dd, 2H), 7.61 (tt, 1H), 7.50 (tt, 2H), 4.93-5.07 (m, 6H), 3.73 (dt, 1H), 2.88 (dddd, 1H), 2.35 (dddd, 1H), 1.92-2.10 (m, 17H), 1.67 (bs, 6H), 1.58-1.65 (overlapping broad singlets, 12H), 1.56 (bs, 3H), 1.20 (d, 3H).

### Example 11. Squalene Phenyl Sulfone Method 2

An oven dried 250-mL three necked round bottom flask and pressure equalizing addition funnel were assembled and cooled under nitrogen. The flask was charged with farnesyl phenyl sulfone (3.7 g, 10.7 mmol), farnesyl chloride (2.9 g, 80% pure, 10.15 mmol), 40 mL tetrahydrofuran and copper (I) iodide (0.23 g, 1.2 mmol). The suspension was cooled in a dry ice/acetonitrile bath at a temperature of approximately −38° C. A solution of potassium tert-butoxide in 25 mL tetrahydrofuran was added to the mixture by dropwise addition and stirring of the cooled reaction was continued for 2 hours. After 3 additional days of stirring at room temperature, most of the solvent was removed by rotary evaporation. The residue was diluted with 100 mL water and extracted with first 100 mL ethyl acetate and then 50 mL ethyl acetate. The combined organic extracts were concentrated to give 6.3 g brown oil containing particulates. The product was purified by silica gel chromatography using a 10% ethyl acetate/heptane to 20% ethyl acetate/heptane step gradient resulting in 3.8 g desired product (68% yield).

### Example 12. Squalene

Squalene phenyl sulfone (2.00 g, 3.66 mmol) was placed in an oven dried 250-ml round bottomed flask equipped with a magnetic stirrer and flushed with argon. Dry tetrahydrofuran (50 mL) was added followed by 1,3-bis(diphenylphosphino)propane palladium(II) dichloride (0.105 g, 0.178 mmol), and the stirred mixture was cooled to −78° C. Lithium triethylborohydride (14.6 ml, 1.0 M in tetrahydrofuran, 14.6 mmol) was added over 1.5 hours and the mixture was stirred at −78° C. for an additional 0.5 hours, and at room temperature for an additional 48 hours. Thin layer chromatography showed that the reaction had completed. Methanol was added until gas evolution ceased, and the tetrahydrofuran was removed under reduced pressure. The residual oil was extracted with ether, water, and saturated sodium chloride. The organic phase was dried over magnesium sulfate, filtered, and concentrated under reduced pressure. The product was further purified by resuspending in hexanes and filtering through silica gel to remove some brown color, yielding squalene as a colorless oil (1.33 g, 88.7%). Proton NMR: 5.06-5.18 (m, 6H), 1.94-2.13 (m, 20H), 1.68 (bs, 6H), 1.60 (bs, 18H).

1. A method for preparing a compound of formula (I) having the structure: 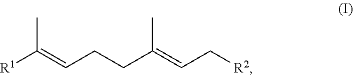
the method comprising:
forming a first reaction mixture comprising a compound of formula NHR³R⁴, a reagent comprising an alkali metal, and a compound of formula (II): 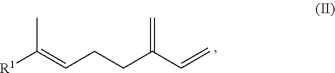
under conditions sufficient to form an amine compound of formula (I) having the structure: 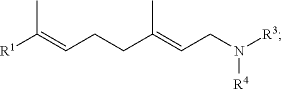
and
forming a second reaction mixture comprising a chloroformate and the amine compound of formula (I), under conditions sufficient to form a chloride compound of formula (I) having the structure: 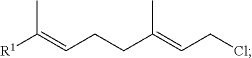
wherein R¹ is selected from the group consisting of C₂₋₁₈ alkyl and C₂₋₁₈ alkenyl; wherein R² is selected from the group consisting of NR³R⁴, halogen, OH, —OC(O)R⁵, and —SO₂—R⁵; wherein R³ and R⁴ are each independently C₁₋₆ alkyl; and wherein R⁵ is selected from the group consisting of C₁₋₆ alkyl, C₃₋₁₀ cycloalkyl, C₃₋₈ heterocycloalkyl, C₆₋₁₂ aryl, and C₅₋₁₂ heteroaryl.

2. The method of claim 1, wherein R³ and R⁴ are each ethyl.

3. The method of claim 1, wherein the alkali metal is sodium or lithium.

4. The method of claim 3, wherein the reagent comprises an alkyllithium compound or an aryllithium compound.

5. The method of claim 3 wherein the reagent comprises n-butyllithium.

6. The method of claim 1, wherein the first reaction mixture further comprises isopropyl alcohol or styrene.

7. The method of claim 1, wherein the chloroformate is isobutyl chloroformate.

8. The method of claim 1, further comprising:
forming a third reaction mixture comprising the chloride compound of formula (I) and a compound of formula (III): 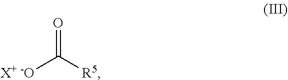
under conditions sufficient to form an ester compound of formula (I) having the structure: 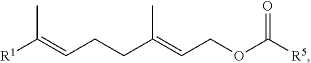
wherein X is an alkali metal.

9. The method of claim 8, wherein the third reaction further comprises a crown ether.

10. The method of claim 8, further comprising:
forming a fourth reaction mixture comprising a strong base and the ester compound of formula (I) under conditions sufficient to form an alcohol compound of formula (I) having the structure:

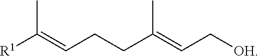

11. The method of claim 10, wherein the strong base comprises sodium hydroxide or potassium hydroxide.

12. The method of claim 1, further comprising:
forming a third reaction mixture comprising a benzenesulfonate, a quaternary ammonium salt, and the chloride compound of formula (I), under conditions sufficient to form a sulfone compound of formula (I) having the structure:

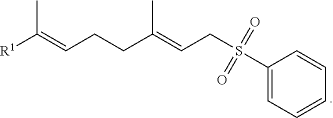

13. The method of claim 12, wherein the benzenesulfonate is sodium benzenesulfonate.

14. The method of claim 12, wherein the quaternary ammonium salt is tetrabutylammonium chloride.

15. The method of claim 12, further comprising:
forming a fourth reaction mixture comprising a strong base, the chloride compound of formula (I), and the sulfone compound of formula (I), under conditions sufficient to form a compound of formula (IV) having the structure: 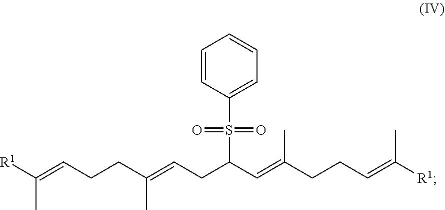
and
forming a fifth reaction mixture comprising a reducing agent, a palladium catalyst, and a compound of formula (IV), under conditions sufficient to form a compound of formula (V) having the structure:

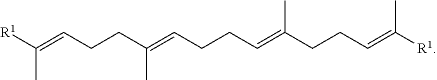

16. The method of claim 15, wherein the fourth reaction mixture further comprises a copper catalyst.

17. The method of claim 16, wherein the copper catalyst comprises copper iodide.

18. The method of claim 15, wherein the strong base comprises potassium tert-butoxide or sodium hydride.

19. The method of claim 15, wherein the reducing agent comprises a borohydride reducing agent.

20. The method of claim 15, wherein the reducing agent comprises lithium.

21. The method of claim 19, wherein the reducing agent is lithium triethylborohydride.

22. The method of claim 15, wherein the palladium catalyst comprises palladium chloride.

23. The method of claim 22, wherein the palladium catalyst comprises [1,2-bis(diphenylphosphino)propane]dichloropalladium(II).

24. The method of claim 1, wherein the compound of formula (II) has the structure:

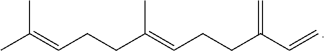

25. The method of claim 1, further comprising:
preparing the compound of formula (II) by a process comprising culturing a microorganism using a carbon source.

26. The method of claim 25, wherein the carbon source is derived from a saccharide.

27. The method of claim 1, wherein the amine compound of formula (I) has the structure:

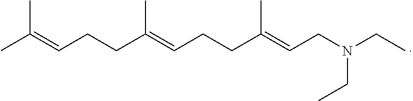 [SMILES: CCN(CC)C/C=C(\C)CC/C=C(\C)CCC=C(C)C]

28. The method of claim 1, wherein the chloride compound of formula (I) has the structure:

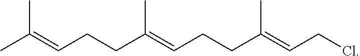 [SMILES: CC(C)=CCC/C(C)=C/CC/C(C)=C/CCl]

29. The method of claim 8, wherein the ester compound of formula (I) has the structure:

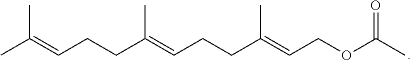 [SMILES: CC(=O)OC/C=C(\C)CC/C=C(\C)CCC=C(C)C]

30. The method of claim 10, wherein the alcohol compound of formula (I) has the structure:

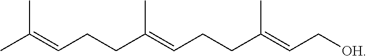 [SMILES: CC(C)=CCC/C(C)=C/CC/C(C)=C/CO]

31. The method of claim 12, wherein the sulfone compound of formula (I) has the structure:

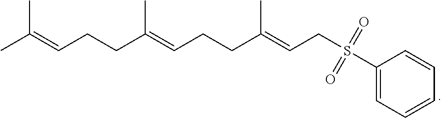 [SMILES: CC(C)=CCC/C(C)=C/CC/C(C)=C/CS(=O)(=O)c1ccccc1]

32. The method of claim 15, wherein the compound of formula (V) has the structure:

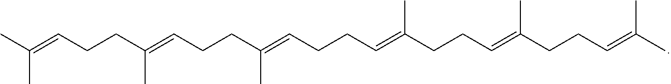 [SMILES: CC(C)=CCC/C(C)=C/CC/C(C)=C/CC/C=C(\C)CC/C=C(\C)CCC=C(C)C]

33. A composition comprising one or more farnesene derivatives prepared using the method of claim 1.

34. The composition of claim 33, comprising from 0.1 wt % to 3 wt % (2Z,5E)-farnesol relative to the total amount of the one or more farnesene derivatives in the composition.

35. The composition of claim 33, comprising from 0.1 wt % to 99.9 wt % (E,E)-farnesol relative to the total amount the one or more farnesene derivatives in the composition.

36. The composition of claim 32, comprising from 0.1 wt % to 99.9 wt % farnesyl acetate relative to the total amount of the one or more farnesene derivatives in the composition.

37. The composition of claim 32, comprising from 0.1 wt % to 99.9 wt % squalene relative to the total amount of the one or more farnesene derivatives in the composition.

38. The composition of claim 37, further comprising an antigen.
